(19) United States
(12) Patent Application Publication
(10) Pub. No.: US 2025/0352039 A1
(43) Pub. Date: Nov. 20, 2025

(54) DISTAL TIPS FOR MEDICAL DEVICES

(71) Applicant: Boston Scientific Scimed, Inc., Maple Grove, MN (US)

(72) Inventors: Sean POWELL, Holden, MA (US); Matthew Robert JAGELSKI, Milford, MA (US); Diana WALLET, Brighton, MA (US)

(73) Assignee: Boston Scientific Scimed, Inc., Maple Grove, MN (US)

(21) Appl. No.: 19/206,895

(22) Filed: May 13, 2025

Related U.S. Application Data
(60) Provisional application No. 63/647,799, filed on May 15, 2024.

(51) Int. Cl. A61B 1/00

(52) U.S. Cl. CPC A61B 1/0008; A61B 1/00137

(57) ABSTRACT

A distal tip of a medical device may comprise a proximal portion a distal portion. Each of a first side surface, a second side surface, a top surface, and a bottom surface may extend along the proximal portion and the distal portion and join together at a distalmost end of the distal tip. A curved shape of the distal portion may be sharper than a hemisphere.

### CROSS-REFERENCE TO RELATED APPLICATIONS

This application claims the benefit of priority to U.S. Provisional Application No. 63/647,799, filed on May 15, 2024, which is incorporated by reference herein in its entirety.

### TECHNICAL FIELD

Various aspects of this disclosure relate generally to distal tips for medical devices. In particular, aspects of this disclosure pertain to distal tips for duodenoscopes, among other aspects.

### BACKGROUND

Duodenoscopes may include a handle and a sheath or shaft, and the shaft may be insertable into a body lumen of a subject. The sheath may terminate in a distal tip portion, which may include features, such as optical elements (e.g., camera, lighting, etc.), air/water outlets, and working channel openings. An elevator may be disposed at a distal tip and may be actuatable in order to change an orientation of a medical device/tool passed through the working channel. For example, the elevator may be pivotable or otherwise movable. During an endoscopic retrograde cholangiopancreatography (ERCP) procedure, an operator may insert a shaft of a duodenoscope into a body lumen and navigate the distal tip to a procedure site. Such body lumens may have small sizes, which may pose challenges to advancement of conventional devices.

### SUMMARY

Aspects of the disclosure relate to distal tips of medical devices that have a decreased size and a bullet-shaped distal end. For example, the distal end may have a more gradual taper, a more pointed shape, and/or a less blunt shape than conventional devices. Each of the aspects disclosed herein may include one or more of the features described in connection with any of the other disclosed aspects.

In an aspect, a distal tip of a medical device may comprise a proximal portion and a distal portion. Each of a first side surface, a second side surface, a top surface, and a bottom surface may extend along the proximal portion and the distal portion and join together at a distalmost end of the distal tip. A curved shape of the distal portion may be sharper than a hemisphere.

Any of the aspects disclosed herein may include any of the following features, alone or in any combination. The distalmost end may be curved and sharper than a hemisphere of diameter equal to the diameter of the distal tip. At least some of the surfaces of the distal portion have a shape that is a portion of an ogive, a portion of a paraboloid, or a portion of an ellipse. Along the distal portion, the first side surface, the second side surface, and the bottom surface may have approximately a same curvature. Along the distal portion, the top surface may have a different curvature from the first side surface, the second side surface, and the bottom surface. A chamfer may extend between the top surface and at least one of the first side surface, the second side surface, or the bottom surface. When viewing the distal tip from the front (viewing the distalmost end, facing proximally), less than or equal to than 50% of the total visible surface area of the distal tip may have a surface with a normal line that is less than or equal to 45 degrees with respect to central a longitudinal axis of the distal tip The distal tip may include a flexible removable cap. The flexible removable cap may include a proximal portion and a distal portion, and the distal portion may protrude radially outward relative to the proximal portion. A plurality of scalloped edges may extend between the proximal portion and the distal portion. The flexible removable cap may be removable while the distal tip is in situ. The flexible removable cap may include a dissolvable, digestible, or passable material. The distal portion may have a partially hemispherical shape. An overall profile of the distal portion may be symmetrical about a plane extending along central longitudinal axis of the distal tip. The plane may extend from a top surface of the distal portion to a bottom surface of the distal portion.

In another aspect, a distal tip of a medical device may comprise: a distal tip body; and a flexible removable cap. The flexible removable cap may include: a proximal portion; and a distal portion that protrudes radially outward relative to the proximal portion. The distal portion may taper inwardly to a distalmost end of the flexible removable cap.

Any of the aspects disclosed herein may include any of the following features, alone or in any combination. A plurality of scalloped edges may extend between the proximal portion and the distal portion. The flexible removable cap may be entirely distal of an imaging device of the distal tip. An entirety of the flexible removable cap may be distal to the imaging device. The flexible removable cap may be removable while the distal tip is in situ. The cap may include a dissolvable, digestible, or passable material.

In another example, a distal tip of a medical device may comprise a proximal portion; and a distal portion. Each of a first side surface, a second side surface, a top surface, and a bottom surface may extend along the proximal portion and the distal portion and join together at a distalmost end of the distal tip. When viewing the distalmost end and facing proximally, less than or equal to than 50% of the total visible surface area of the distal tip has a surface with a normal line that is less than or equal to 45 degrees with respect to a central longitudinal axis of the distal tip.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict an exemplary medical device.

FIGS. 2A-2G depict aspects of an exemplary distal tip for use with the medical device of FIGS. 1A and 1B.

FIGS. 3A-3B depict another exemplary distal tip.

FIGS. 4A-4B depict a further exemplary distal tip.

FIGS. 5A-5B depict an alternative exemplary distal tip.

### DETAILED DESCRIPTION

Distal tips of medical devices, such as duodenoscopes, may be difficult to navigate through small body lumens (e.g., body lumens of pediatric subjects or body lumens with strictures). The disclosed distal tips may have a smaller diameter/width than conventional distal tips, so as to facilitate navigation through smaller anatomies. Furthermore, the distal ends of the disclosed distal tips may be less blunt than conventional distal tips, to facilitate navigation, particularly through strictures.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. In some drawings, arrows labeled “P” and “D” indicate proximal and distal directions, respectively. The term “exemplary” is used in the sense of “example,” rather than “ideal.” The term “approximately,” or like terms (e.g., “substantially”), includes values+/−10% of a stated value.

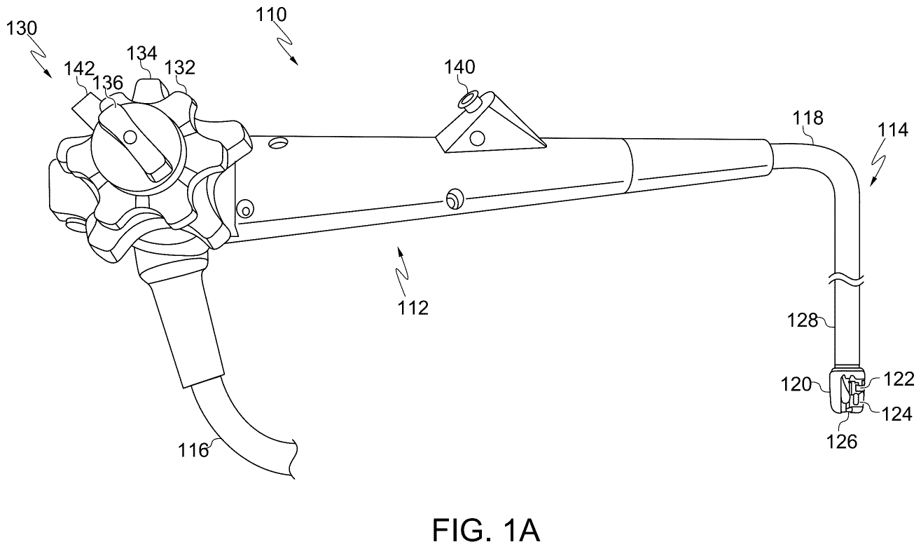

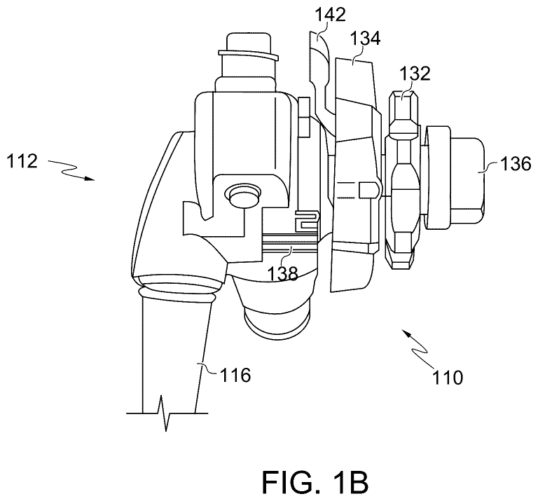

FIG. 1A depicts an exemplary medical device 110 having a handle 112 and an insertion portion 114. FIG. 1B shows a proximal end of handle 112. Medical device 100 may also include an umbilicus 116 for purposes of connecting medical device 10 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 114 may include a sheath or shaft 118 and a distal tip 120. Distal tip 120 may include an imaging device 122 (e.g., a camera) and a lighting element 124 (e.g., a light emitting diode (LED) or an optical fiber). Although the term “lighting element” is used herein, it will be appreciated that the term “lighting element” may include a plurality of lighting elements (e.g., a plurality of LEDs or optical fibers). Distal tip 120 may be side-facing. That is, imaging device 122 and lighting element 124 may face radially outward, perpendicularly, approximately perpendicularly, or otherwise transverse to a longitudinal axis of shaft 118 and distal tip 120 (e.g., at an angle of approximately 80 degrees to an angle of approximately 110 degrees). However, the disclosure is not limited to such an arrangement.

Distal tip 120 may also include an elevator 126 for changing an orientation of an accessory device or a tool inserted in a working channel of medical device 110. Elevator 126 may alternatively be referred to as a swing stand, pivot stand, raising base, or any suitable other term. Elevator 126 may be pivotable via, e.g., an actuation wire or another control element that extends from handle 112, through shaft 118, to elevator 126.

A distal portion of shaft 118 that is connected to distal tip 120 may have a steerable section 128. Steerable section 128 may be, for example, an articulation joint. Shaft 118 and steerable section 128 may include a variety of structures that are known or may become known in the art.

Handle 112 may have one or more actuators/control mechanisms 130. One or more of control mechanisms 130 may provide control over steerable section 128. One or more of control mechanisms 130 may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include one or more locking mechanisms 136a, 136b (e.g., knobs or levers) for preventing steering and/or braking of steerable section 128 in at least one of an up, down, left, or right direction. Handle 112 may include an elevator control lever 138 (see FIG. 1B). Elevator control lever 138 may raise and/or lower elevator 126, via connection between lever 138 and an actuating wire that extends from lever 138, through shaft 118, to elevator 126. A port 140 may allow passage of a tool through port 140, into a working channel of the medical device 110, through shaft 118, to distal tip 120.

In use, an operator may insert at least a portion of shaft 118 into a body lumen of a subject. Distal tip 120 may be navigated to a procedure site in the body lumen. For example, the operator may push or urge distal tip 120 distally in order to advance distal tip 120 through the body lumen. The operator may insert an accessory device (not shown) into port 140, and pass the accessory device through shaft 118 via a working channel to distal tip 120. The accessory device may exit the working channel at distal tip 120. The user may use elevator control lever 138 to raise elevator 126 and angle the accessory device toward a desired location (e.g., a papilla of the pancreatico-biliary tract). The user may use the accessory device to perform a medical procedure.

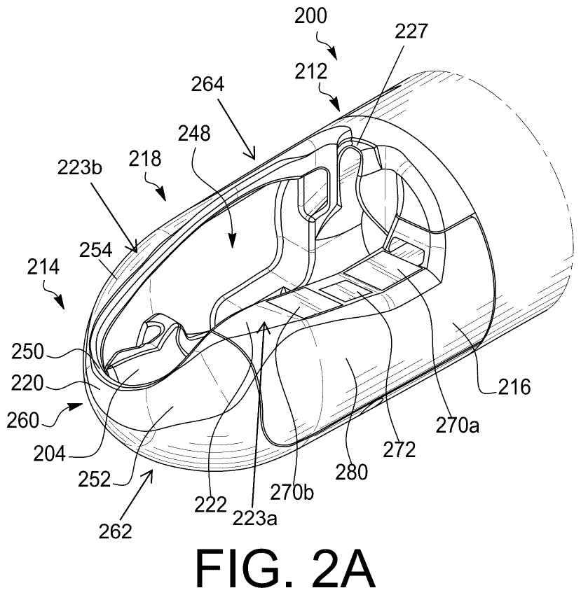

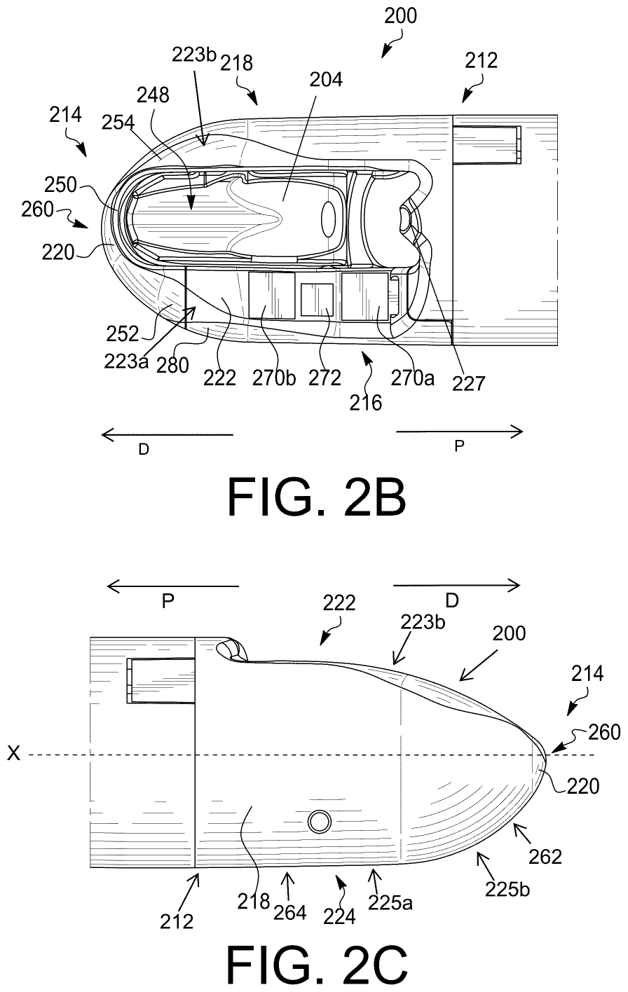

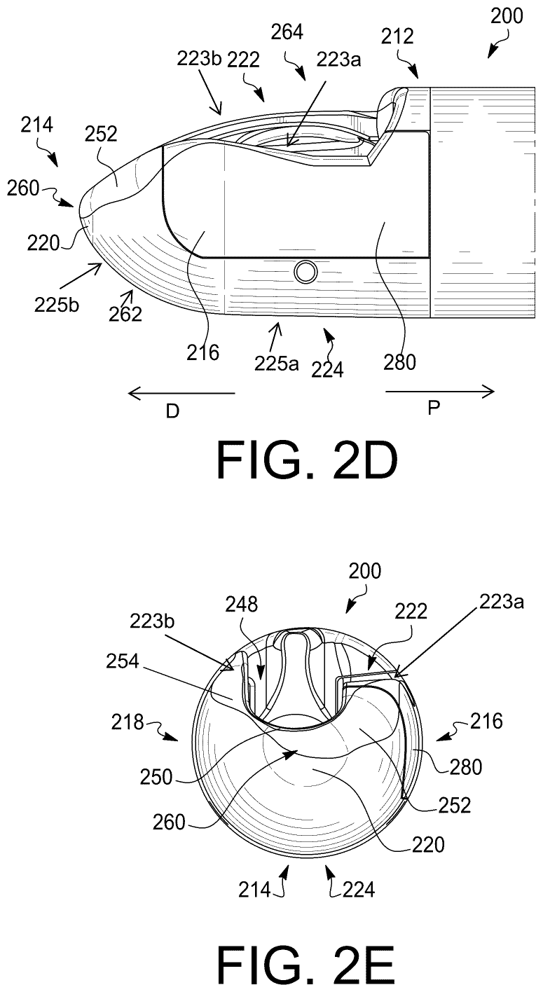

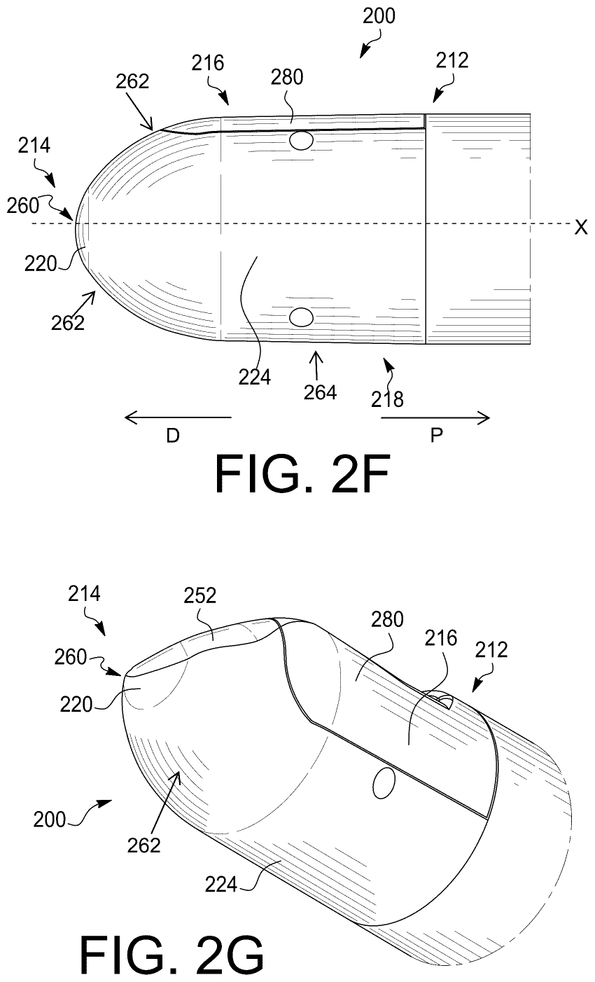

FIGS. 2A-2G depict an exemplary distal tip 200, which may be used in conjunction with medical device 110 in place of distal tip 120. Distal tip 200 may have any of the features of distal tip 120. FIG. 2A is a perspective view of distal tip 200. FIG. 2B shows a top view of distal tip 200. FIGS. 2C and 2D show side views of distal tip 200. FIG. 2E shows a front (proximally-facing) view of distal tip 200. FIG. 2F is a bottom view of distal tip 200. FIG. 2G is another perspective view of distal tip 200.

Distal tip 200 may extend between a proximal end 212 and a distal end 214. A distal surface 220 may be disposed at distal end 214. Distal tip 200 may have a first side surface 216 and a second side surface 218. First side surface 216 may be on an opposite side of distal tip 200 from second side surface 218. First side surface 216 and second side surface 218 may be lateral side surfaces of distal tip 200.

Distal tip 200 may have a top surface 222 and a bottom surface 224. Top surface 222 may be on an opposite side of distal tip 200 from bottom surface 224. Top surface 222 of distal tip 200 may be on a side/portion of distal tip 200 that includes surfaces, openings, or other elements (described in further detail below) for receiving electronics. The electronics may include an imaging device 272 and lighting elements 270a, 270b. Further details of imaging device 272 and lighting elements 270a, 270b are described below. Top surface 222 may also include an opening 248 to accommodate elevator 204. As elevator 204 rotates/pivots, a range of motion of an accessory device or tool (not shown) may pass through various portions of opening 248.

Side surfaces 216, 218, top surface 222, and bottom surface 224 may come together at or converge to form distal surface 220. Although distal surface 220 is described herein, it will be appreciated that distal tip 200 may be thought of as lacking distal surface 220 and instead having side surfaces 216, 218, top surface 222 that extend to and join together at a distalmost end 260 of distal tip 200.

Distal end 214 may include a recess 250 that is in communication with opening 248. An accessory device extending along elevator 104, when elevator 104 is in a lowered configuration (as shown in the Figures), may extend through (and may optionally contact) recess 250. As shown particularly in FIG. 2A, recess 250 may have rounded surfaces. A proximal portion of top surface 222 may include a boss 227 (labeled in FIGS. 2A and 2B) for interacting with and, in some examples, locking an accessory device extending along elevator 204 when elevator 204 is in a raised configuration.

Generally, intersections between distal surface 220, first side surface 216, second side surface 218, top surface 222, and bottom surface 224 may be rounded without sharp corners formed at junctions between the various surfaces. Such rounded junctions may provide for smoother interactions between distal tip 200 and surfaces of a subject's body lumen. As described in further detail below, distal tip 200 may have a bullet or torpedo shape, for example, with a relatively more pointed (e.g., more acute or sharper angled/curved) tip than conventional distal tips of duodenoscopes or other scope devices. Thus, distal tip 200 may more easily navigate narrow passageways, such as strictures. Shapes of the portions of distal tip 200 described herein may be particularly chosen to balance accommodation of elements of distal tip 200 (e.g., elevator 204 and/or electronic elements) while providing a shape and size of distal tip 200 such that distal tip 200 advantageously navigates small spaces within a body lumen and/or may advance through strictures or other narrowed body lumens. Distal tip 200 may have an overall smaller size than conventional duodenoscope distal tips, in order to treat pediatric subjects and/or subjects with narrowed body lumens or portions of body lumens.

Distal tip 200 may have a distalmost tip or end 260. As shown best in the side views of FIGS. 2C and 2D, a distal portion 262 of distal tip 200 may have a shape similar to an ogive; however, distalmost end 260 may be blunted (curved) rather than pointed. As compared with a hypothetical distal tip with a hemispherical distal portion (a hemispherical shape with a diameter equal to the diameter/width of the distal tip 220/shaft 118), distal portion 262, including distalmost end 260 may have a sharper curve profile. In other words, distalmost end 260 may have a sharper curve than a hemisphere of equal diameter to that of distal tip 220/shaft 118. In some examples, distalmost end 260 may have an at least partially hemispherical shape, and other portions of distal portion 262 may not be hemispherical. A shape of distalmost end 260 and distal portion 262 may inhibit perforations in a subject's anatomy. Smoothly curved surfaces of distal portion 262 and smooth transitions between distal portion 262 and a proximal portion 264 of distal tip 200 may help to ease a transition of tissue along surfaces of distal tip 200 and may spread open tight (e.g., small) orifices.

As shown particularly in FIGS. 2C and 2D, bottom surface 224 and top surface 222 may have different curve profiles or shapes, such that a side perimeter of distal tip (including, e.g., distal portion 262) may be asymmetrical. For example, bottom surface 224 may have a proximal portion 225a along proximal portion 264 of distal tip 200 and a distal portion 225b along distal portion 262 of distal tip 200. Proximal portion 225a may have a shape that is approximately a portion of a cylinder or a cone. When viewed in side profile, as in FIGS. 2C and 2D, a bottom perimeter of proximal portion 264 may be substantially straight or straight. In some examples, the bottom perimeter (in side profile) of proximal portion 264 may taper slightly radially inward, toward a central longitudinal axis X (labeled in FIGS. 2C and 2F) of distal tip 200, moving in a distal direction. The central longitudinal axis of distal tip 200 may be parallel to the proximal/distal directions labeled in some of FIGS. 2A-2G. Proximal portion 225a may have a uniform shape or may have portions with variable shapes.

Distal portion 225b may have a shape that may be similar to a portion of an ellipsoid, a portion of an ogive, and/or a portion of a paraboloid. In other words, when viewed in side profile (FIGS. 2C and 2D), a bottom perimeter of distal portion 262 may have a shape similar to a portion of a parabola, a portion of an ellipse, or another curve. Distal portion 225b may taper radially inward, moving in a distal direction, toward the central longitudinal axis of distal tip 200. Distal portion 225b may have a uniform shape or may have portions with variable shapes. A shape of proximal portion 225a and distal portion 225b of bottom surface 224 may accommodate elevator 204 while also providing for an increased sharpness of distal portion 262.

Top surface 222 may be laterally asymmetrical. A first portion 223a of top surface 222, adjacent to first side surface 216, may receive imaging device 272 and lighting elements 270a, 270b. As shown in FIG. 2B, imaging device 272 may be disposed between lighting elements 270a, 270b. A line extending in the proximal/distal directions may intersect imaging device 272 and lighting elements 270a, 270b. A chamfer 252 (e.g., a transition surface) may extend between top surface 222 and distal surface 220 and/or first side surface 216. The term “chamfer” as used herein is not limiting to angles of 45 degrees and encompasses transitions of varying angles. Chamfer 252 may help to increase a “sharpness” of distal portion 262/distalmost end 260 and/or may transition between different curvatures of top surface 222, distal surface 220, and/or first side surface 216.

A second portion 223b of top surface 222, adjacent to second side surface 218, may extend between opening 248 and second side surface 218. Second portion 223b may have a chamfer 254 extending between top surface 222, side surface 218, and/or distal surface 220. As shown particularly in FIGS. 2B-2E, chamfers 252 and 254 may not have the same shape (and may not be symmetrical). Chamfer 252 and 254 may be portions of a single, larger chamfer, as shown in FIG. 2E or may be separate from one another. As shown in FIG. 2E, particularly, chamfers 252 and 254 may surround recess 250. Chamfer 252 may extend further distally and/or further toward bottom surface 224 than chamfer 254. Chamfer 252 may also be laterally wider than chamfer 252. Chamfer 252 may overall be larger than chamfer 254. The shapes of chamfers 252, 254 may accommodate the respective elements of first portion 223a and second portion 223b. For example, a presence of imaging device 272 and lighting elements 270a, 270b may require a greater transitional area to be provided by chamfer 252.

As shown in FIG. 2D, particularly, a middle or intermediate portion of first portion 223a (a portion of first portion 223a accommodating imaging device 272 and lighting elements 270a, 270b) may be recessed with respect to a corresponding portion of second portion 223b. In other words, a middle or intermediate portion of second portion 223b may extend higher in FIG. 2D than a middle or intermediate portion of first portion 223a. As shown in FIGS. 2C and 2D, distally of imaging device 272 and lighting elements 270a, 270b, first portion 223a and second portion 223b may have similar or the same heights.

As shown in FIG. 2C, an overall profile of second portion 223b (excluding chamfer 254) may be similar to a portion of an ellipsoid, a portion of an ogive, and/or a portion of a paraboloid. In other words, when viewed from the side, as in FIG. 2C, an outer perimeter of second portion 223b may have a shape similar to a portion of a parabola, a portion of an ellipse, or another curve. As compared to bottom surface 224, second portion 223b may begin to taper radially inward at a more proximal location. For example, second portion 223b may taper along proximal portion 264 of distal tip 200. As compared to distal portion 225b of bottom surface 224, second portion 223b may have a larger radius of curvature (may curve more gradually). The distal portion of first portion 223a may have approximately the same radius of curvature as second portion 223b.

As shown particularly in FIGS. 2B and 2F, an overall profile of distal tip 200 may be approximately symmetrical about a plane extending along central longitudinal axis X (labeled in FIG. 2F) and between top surface 222 and bottom surface 224 (i.e., a plane extending along central longitudinal axis X and into/out of the page of FIG. 2F). In other words, an outer perimeter of distal tip 200, when viewed from the top or the bottom (FIGS. 2B and 2F) may be symmetrical about a plane extending along central longitudinal axis X and between top surface 222 and bottom surface 224. The perimeter of distal portion 262, when viewed from the top or bottom, may be similar to a parabola, a portion of an ellipse, or may be another curved shape.

When distal tip 200 is viewed from above or below, a perimeter of proximal portion 264 may be approximately straight along each of side surfaces 216 and 218. In some examples, the perimeter along each of side surfaces 216 and 218 may taper inward slightly toward longitudinal axis X, moving in a distal direction. Alternatively, the perimeter along each of side surfaces 216 and 218 may be parallel to longitudinal axis X or taper outwardly in a distal direction.

A curvature (e.g., a curve profile) of the side perimeters of distal portion 262 (when viewing distal tip 200 from the top or the bottom as in FIGS. 2B and 2F) may be similar to or the same as a curvature of the bottom perimeter of distal portion 262 (when viewing distal tip 200 from the side as in FIGS. 2C and 2D). In contrast, a curvature of the top perimeter of distal tip 200 may differ (when viewing distal tip 200 from the side as in FIGS. 2C and 2D), as described above. Thus, as described above, chamfers 252 and 254 may transition between the portions of different curvature profiles.

In an example, side surfaces 216, 218 and bottom surface 224 may all have a same curvature shape on distal portion 262. Top surface 222 may have a different curvature shape or profile. Chamfers 252, 254 may extend between top surface and side surfaces 216, 218, bottom surface 224, and/or distal surface 220 in order to transition between the different curvature shapes. An overall shape of distal portion 262 may be similar to that of an ogive or paraboloid, with a top portion of distal portion 262 (having top surface 222) having an irregular shape. Similarly, side surfaces 216, 218 and bottom surface 224 may all have a same curvature shape on proximal portion 264. Top surface 222 may have a different curvature shape or profile on proximal portion 264. Proximal portion 264 may have an overall tubular shape, with a top surface of proximal portion 264 (having top surface 222) having an irregular shape.

Imaging device 272 and lighting elements 270a, 270b may be housed within a module 280. Module 280 may form a portion of an outer surface of distal tip 200, but may be manufactured as a separate element from a remainder of distal tip 200 and inserted into distal tip 200. For example, module 280 may form a portion of first side surface 216. Module 280 may include portions of chamfer 252. A shape of module 280 may be continuous with a shape of adjacent portions, such that distal tip 200 has a continuous outer shape.

As compared with conventional devices, angles of surfaces of distal tip 200 (and particularly distal portion 262) with respect to longitudinal axis X may be generally smaller. For example, in conventional duodenoscopes, a distal surface of a conventional distal tip may have an approximately 90 degree angle with respect to a central longitudinal axis of the duodenoscope. In contrast, when viewing distal tip 200 from the front (FIG. 2E), a relatively small proportion of distal tip 200 may have surfaces with normal lines that have angles of less than 45 degrees with respect to longitudinal axis X (extending into and out of the page in FIG. 2E). For example, less than or equal to 50% of the total visible surface area of distal tip 200 in the view of FIG. 2E (the front view, facing proximally) may have normal lines that are less than or equal to 45 degrees with respect to central longitudinal axis X.

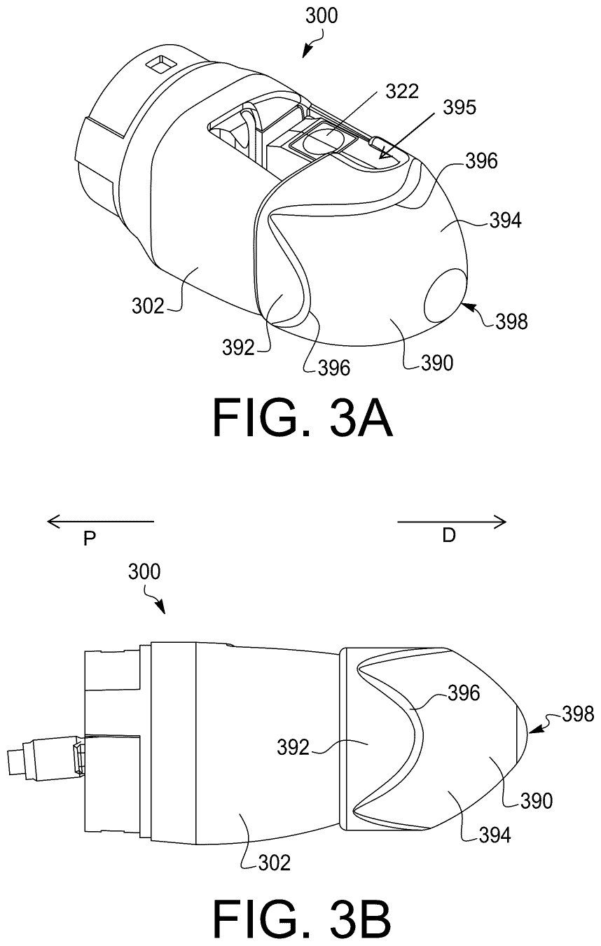

FIGS. 3A and 3B show an alternative distal tip 300 that may have any of the features of distal tips 120, 200, unless otherwise stated. Distal tip 300 may have a distal tip body 302 and a cap 390. Cap 390 may be a disposable, soft distalmost portion of distal tip 300. Cap 390 may be removably positioned over distal tip body 302 and may be removed when distal tip 300 is in situ within a subject's body lumen. Cap 390 may include a dissolvable, digestible, or passable material. Cap 390 may be used with distal tips of conventional medical devices, in some examples. In alternatives, cap 390 may be overmolded or otherwise manufactured onto distal tip body 302.

In examples, cap 390 may be formed (e.g., molded) from a soft, sterile material. The softness of cap 390 may enable cap 390 to have a more pointed/sharp profile than would be feasible for a rigid distal tip, because the soft cap 390 may be less likely to cause damage to a body lumen. Cap 390 may be affixed to distal tip body 320 using, for example, a biologically safe adhesive. In some examples, cap 390 may snap onto rigid portions of distal tip 300. Additionally or alternatively, an elevator of distal tip 300 (not shown but having any of the properties of elevators 126, 204) may be positioned over a feature of cap 390 to secure cap 390 in place until the elevator is actuated.

In examples, cap 390 may be disposed entirely distally of an imaging device 322 (in other words, an entirety of cap 390 may be distal of imaging device 322). In some alternative examples, cap 390 may include a proximal sleeve (not shown) that may be rolled over or otherwise positioned around a rigid portion of distal tip body 302. The proximal sleeve (and any other portions of cap 390) may be formed of a transparent material to allow for viewing by imaging device 322 (having any of the features of imaging devices 122 or 272). Additionally or alternatively, the proximal sleeve may have a hole or other opening that may be positioned over imaging device 322.

In some examples, cap 390 may be removable from distal tip body 302 during a procedure. In such examples, cap 390 may have a retrieval element (e.g., a cord or a tab) to help allow for cap 390 to remain tethered to distal tip body 302 and/or be retrieved by an operator. Additionally or alternatively, cap 390 may be formed of a low-sugar gelatin-like gummy material that may be safely discarded and/or consumed by the subject, a bioabsorbable material which will naturally break down in a subject, or a soft material which will naturally be passed by a subject.

Cap 390 may have a proximal portion 392 and a distal portion 394. Distal portion 394 may be raised or otherwise protrude outwardly (e.g., radially outwardly) with respect to proximal portion 392. Proximal portion 392 may form a sleeve that helps to secure cap 390 to distal tip body 302. Proximal portion 392 may include one or more notches or cutouts 395 to accommodate electronic elements (e.g., lighting elements) or other elements of distal tip 300. Distal portion 394 may protrude outward, so that distal portion 394 may modify an outer profile of distal tip body 302 to a desired shape.

For example, distal portion 394 may have an ogive, paraboloid, and or partial-ellipsoid shape. Overall, distal portion 392 may have a bullet or torpedo shape. Distal portion 394 may have a distalmost end 398, which may be curved but relatively sharper/more pointed than a hemisphere of equal diameter to the diameter of distal tip 300. A longitudinal axis extending through a center of distalmost end 398 may be coaxial with a central longitudinal axis extending through distal tip 300.

A proximal edge of distal portion 394 may include scallops 396 (a plurality of scalloped edges). Each of scallops 396 may have a central portion that is further distal of side portions of the respective scallop 396. Scallops 396 may help to provide a smooth and atraumatic transition between a larger cross-section of distal portion 394 and a smaller cross-section of proximal portion 392 and distal tip body 302. Each of scallops 396 may have the same shape. In the example of FIGS. 3A and 3B, cap 390 may include exactly four scallops 396 (e.g., top, bottom, first side, and second side scallops 396). A shape of scallops 396 may be particularly compatible with a shape of distal portion 394. For example, distal portion 394 may be approximately symmetrical about a central longitudinal axis of distal portion 394, and scallops 396 may all have a same shape.

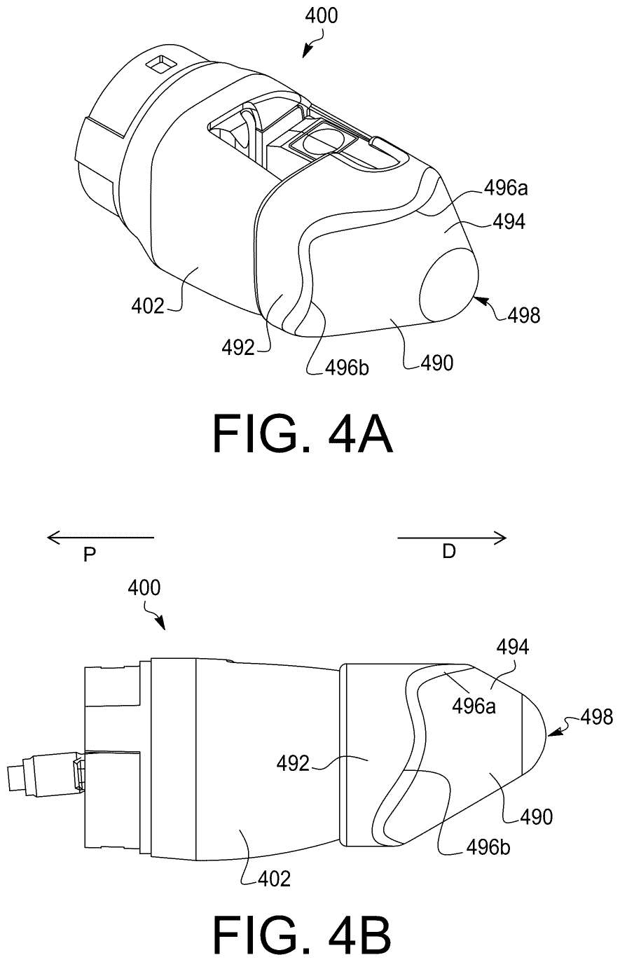

FIGS. 4A and 4B show an alternative distal tip 400 having a distal tip body 402 and a cap 490. Distal tip 400 may have any of the features of distal tip 300, unless otherwise specified. Similar to cap 390, cap 490 may have a proximal portion 492 and a distal portion 494. Distal portion 494 may have a cone shape, which may taper to a distalmost end 498. Distalmost end 498 may have a relatively more pointed, a relatively blunter, or a similar shape to distalmost end 398. A longitudinal axis extending through a center of distalmost end 498 may be parallel to but not coaxial with a central longitudinal axis of distal tip 400. For example, as shown in FIGS. 4A and 4B, distalmost end 498 may be closer to a top side (a side having the lighting/imager) of distal tip 400 and further from an opposite, bottom side of distal tip 400.

As shown particularly in FIG. 4B, cap 490 may have scallops 496a, 496b that have differing shapes. Alternatively, scallops 496a, 496b may have the same shape (e.g., the same shape as scallops 396). Scallop 496a may be on a top surface of cap 490 and may have a similar shape to scallop 396. Scallop 496b may be on a side surface of cap 490. An upper end of scallop 496b (an end closer to a top surface of distal tip 400) may be distal to a bottom end of scallop 496b (an end closer to a bottom surface of distal tip 400). A middle portion of scallop 496b may be distal to the upper end and the bottom end of scallop 496b.

Although only two scallops 496a, 496b are shown in FIGS. 4A and 4B, other sides or portions of cap 490 may have scallops having any of the properties of scallops 496a, 496b. For example, a bottom surface of cap 490 may have a scallop similar to scallop 496a. However, the scallop on the bottom surface of cap 490 may be proximal to scallop 496a. A side of cap 490 opposite to scallop 496b may have a scallop with any of the same properties of scallop 496b. A shape of scallops 496a, 496b, etc. may be particularly compatible with a shape of distal portion 494. For example, scallop 496a may be distal to a scallop on the bottom of cap 490, due to distalmost end 498 being closer to a top side distal tip 400. The shapes of the scallops shown in the Figures may accommodate the different respective angles of the surfaces of cap 490.

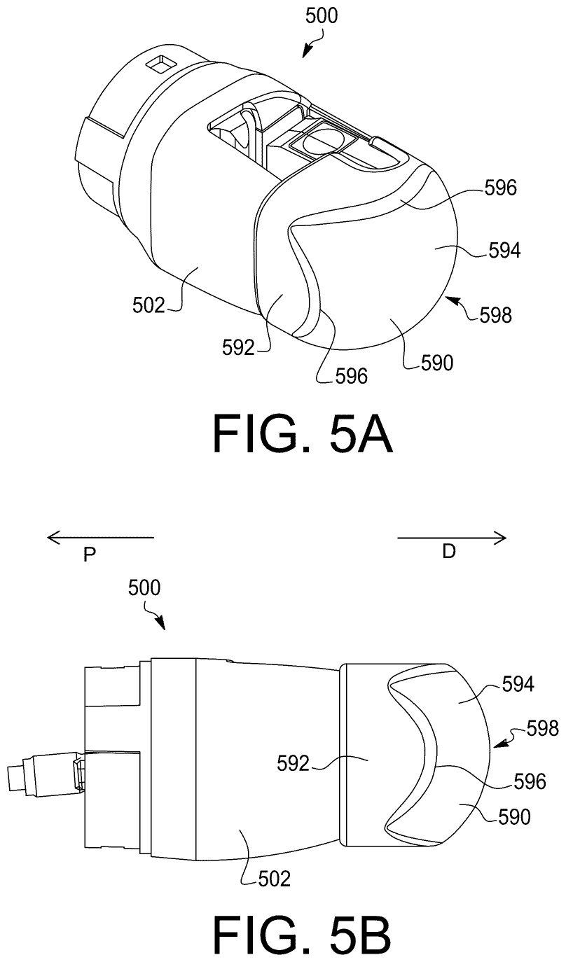

FIGS. 5A and 5B show an alternative distal tip 500 having a distal tip body 502 and a cap 590. Distal tip 500 may have any of the features of distal tips 300 or 400, unless otherwise specified. Similar to caps 390 and 490, cap 590 may have a proximal portion 592 and a distal portion 594. Distal portion 594 may have a blunter shape than distal portions 394, 494. For example, distal portion 594 may have a shape that is similar to a dome or a hemisphere of equal or similar diameter to the distal tip 500. A distalmost end 598 of cap 590 may be more rounded or blunter than distalmost ends 398 and 498. Similar to cap 390, cap 590 may include a plurality of scallops 596 having any of the properties of scallops 396. Alternatively, scallops 596 may have any of the properties of scallops 496a, 496b.

The features of caps 390, 490, 590 may be combined in any suitable manner. For example, shapes of distal portions 394, 494, and 594 may be combined to create shapes that are intermediate of the shapes described. An operator may elect to use a cap 390, 490, 590 that aligns with a profile of a procedure to be performed. For example, cap 590 may be gentler/less likely to cause perforations on more delicate tissue. Cap 490 may be more able to traverse narrowed body lumens and to spread open the narrowed portion of the lumen to allow distal tip 400 to pass through the body lumen. Cap 390 may be an intermediate option that is more able to traverse narrow body lumens than cap 590, but is gentler on tissue than cap 490. The shapes of scallops may 396, 496a, 496b, 596 may be combined in a manner suitable to transition between distal portions 394, 494, 594 and proximal portions 392, 492, 592.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented examples can be combined to achieve a same or similar result as one or more of the disclosed embodiments. Thus, the features of the examples should not be viewed as being mutually exclusive or applicable to any particular distal tip and may be combined in any suitable manner. Accordingly, the invention is not to be considered as limited by the foregoing description.

We claim:

1. A distal tip of a medical device, the distal tip comprising:
a proximal portion; and
a distal portion;
wherein each of a first side surface, a second side surface, a top surface, and a bottom surface extend along the proximal portion and the distal portion and join together at a distalmost end of the distal tip;
wherein a curved shape of the distal portion is sharper than a hemisphere.

2. The distal tip of claim 1, wherein the distalmost end is curved and sharper than a hemisphere of diameter equal to the diameter of the distal tip.

3. The distal tip of claim 1, wherein at least some of the surfaces of the distal portion have a shape that is a portion of an ogive, a portion of a paraboloid, or a portion of an ellipse.

4. The distal tip of claim 1, wherein, along the distal portion, the first side surface, the second side surface, and the bottom surface have approximately a same curvature.

5. The distal tip of claim 4, wherein, along the distal portion, the top surface has a different curvature from the first side surface, the second side surface, and the bottom surface.

6. The distal tip of claim 5, wherein a chamfer extends between the top surface and at least one of the first side surface, the second side surface, or the bottom surface.

7. The distal tip of claim 1, wherein, when viewing the distalmost end and facing proximally, less than or equal to than 50% of the total visible surface area of the distal tip has a surface with a normal line that is less than or equal to 45 degrees with respect to central a longitudinal axis of the distal tip.

8. The distal tip of claim 1, wherein the distal portion includes a flexible removable cap.

9. The distal tip of claim 8, wherein the flexible removable cap includes a proximal portion and a distal portion, wherein the distal portion protrudes radially outward relative to the proximal portion.

10. The distal tip of claim 9, wherein a plurality of scalloped edges extends between the proximal portion and the distal portion.

11. The distal tip of claim 8, wherein the flexible removable cap is removable while the distal tip is in situ.

12. The distal tip of claim 8, wherein the flexible removable cap includes a dissolvable, digestible, or passable material.

13. The distal tip of claim 1, wherein the distal portion has a partially hemispherical shape.

14. The distal tip of claim 1, wherein an overall profile of the distal portion is symmetrical about a plane extending along central longitudinal axis of the distal tip.

15. The distal tip of claim 14, wherein the plane extends from a top surface of the distal portion to a bottom surface of the distal portion.

16. A distal tip of a medical device, the distal tip comprising:
a distal tip body; and
a flexible removable cap, wherein the flexible removable cap includes:
a proximal portion; and
a distal portion that protrudes radially outward relative to the proximal portion, wherein the distal portion tapers inwardly to a distalmost end of the flexible removable cap.

17. The distal tip of claim 16, wherein a plurality of scalloped edges extends between the proximal portion and the distal portion.

18. The distal tip of claim 16, wherein an entirety of the flexible removable cap is distal of an imaging device of the distal tip.

19. The distal tip of claim 16, wherein the flexible removable cap is removable while the distal tip is in situ, and wherein the cap includes a dissolvable, digestible, or passable material.

20. A distal tip of a medical device, the distal tip comprising:
a proximal portion; and
a distal portion;
wherein each of a first side surface, a second side surface, a top surface, and a bottom surface extend along the proximal portion and the distal portion and join together at a distalmost end of the distal tip; and
wherein, when viewing the distalmost end and facing proximally, less than or equal to than 50% of the total visible surface area of the distal tip has a surface with a normal line that is less than or equal to 45 degrees with respect to a central longitudinal axis of the distal tip.
